(19) United States
(12) Patent Application Publication
(10) Pub. No.: US 2008/0096185 A1
(43) Pub. Date: Apr. 24, 2008

(54) Method for Eliminating potentially toxic and/or harmful substances

(75) Inventors: Gerhard Putz, Freiburg (DE); Jurgen Eckes, Freiburg (DE)

Correspondence Address: EDWARDS ANGELL PALMER & DODGE LLP, P.O. BOX 55874, BOSTON, MA, 02205, US

(73) Assignee: Heinrich Wieland

(21) Appl. No.: 11/973,701

(22) Filed: Oct. 10, 2007

Related U.S. Application Data
(62) Division of application No. 10/473,610, filed on Sep. 29, 2003, now abandoned.

(30) Foreign Application Priority Data
Apr. 5, 2001 (DE) 101 17 043.2
Apr. 5, 2002 (EP) PCT/EP02/03800

(51) Int. Cl. C12Q 1/00; C02F 101/00

(52) U.S. Cl. 435/4; 435/262

(57) ABSTRACT

The invention describes a method for eliminating potentially toxic and/or harmful substances, wherein particles which are capable of binding, taking up and/or carrying the toxic and/or harmful substances, are removed from a body fluid in an extracorporeal step or in an extrinsic or exogeneous device.

The present invention concerns a method for eliminating toxic and/or harmful substances from the bodies of humans or animals. It further concerns a method of medical therapy, a kit for medical therapy or research purposes as well as the use of liposomes in the methods or kit. Moreover, the invention concerns a method in which a therapeutic substance is eliminated, reduced or its dosage is controlled by complete or partial elimination, said method being conducted during or after a medical therapy.

Therapeutic agents may possess a cytotoxic or other toxic effect, e.g. medical therapies directed at malignant (tumor) diseases or infections often involve agents that are highly toxic also for healthy tissues or organs of the body. Aside from chemotherapeutic agents and cytostatic agents, this may apply to other anti-cancer agents, antibiotics, antiviral agents, anti-malaria agents, antimycotic agents, interferons, cytokines, etc. These substances are often administered by systemic application, in particular by an intravenous route. Since the binding or uptake of these substances by the cells or at/by an other target site usually shows little selectivity, the therapy is associated not only with the desired effects on the target (target organ), but also displays undesired effects on actually functional and, in some cases, vital cells, tissues or organs of the body. Particularly susceptible to this effect are for instance bone marrow cells with ensuing (in some cases—strong) functional impairment of the production of blood cells; the gastrointestinal tract with ensuing vomiting, diarrhea, malabsorption; inflammations; disturbed hair growth or alopecia; in intertriginous areas (folds of the body) also inflammations of the hair follicles with sweat gland abscesses; ulcerations and inflammations of the internal and/or external mucosa, etc. The liver and kidneys may also be affected in a dose-dependent fashion and with extensive interindividual differences that are difficult to anticipate. The two latter organs are involved not only in the detoxification of noxious substances employed for therapeutic purposes, but also possess elementary significance as “blood and body detoxifiers” and as such should be preserved to the extent possible.

Another problem is the elevated risk of the manifestation of secondary and therapy-associated secondary tumors (e.g. lymphomas), whereby cytostatic therapy may inadvertently cause irreversible damage to the genetic information of the healthy cell. The clinical outcome of this may be the uncontrolled proliferation of the damaged cell and ensuing secondary tumor disease whose emergence may not be completely differentiable from a “natural” course (“random accumulation”), but whose cause must be considered to be very likely therapy-associated.

Certain chemotherapeutic agents are associated with a substantially higher risk of undesired and possible serious “adverse drug effects” (AEs, side effects). Pertinent examples include the cardiotoxicity with ensuing cardiac insufficiency (at increased single or total dosage) after the administration of doxorubicin and daunorubicin, and lung fibrosis on bleomycin and nitrosourea derivatives. The manifestation of an AE may be life-threatening for the patient or cause chronic damage and ensuing massive impairment of the patient's quality of life. By surpassing an upper limit (“total dose”) that is only defined by empirical criteria at this time, due to rather unselective application of the agent and because the residual agent is allowed to remain in the body, the application of some sensible follow-up treatment may be jeopardized even though the tumor would have been “responsive” to the treatment. The present invention attempts to avoid these shortcomings by providing for the administration of the agent to be more selective and easier to control.

Moreover, careful dose adjustment balancing the therapeutic effects against the toxic effects may be indicated and possibly necessitate the limitation to low doses of the respective agent. However, as a result of being limited to low dose agent it may not be possible to establish the threshold concentration required at the target site in the organism in order to render the agent effective. The administration of toxic agents at low dose for a prolonged period of time may also be associated with a risk of eliciting the body's own defense by developing resistance (multi-drug resistance, MDR), see J. Robert: “Multidrug resistance in oncology: diagnostic and therapeutic approaches”, Eur J Clin Invest 29(6) (1999), p. 536-545.

Thus, it was the object of the present invention to improve conventional therapeutic options that are based on potentially toxic agents.

The object is solved by the invention providing a method for eliminating potentially toxic and/or harmful substances, wherein particles, which can bind, take up and/or carry said potentially toxic and/or harmful substances, are removed from a body fluid in an extracorporeal step or in an extrinsic or exogeneous device.

In another aspect, the invention provides a method of medical therapy, in which this specific elimination method is performed following the administration of a therapeutic agent or pharmaceutical composition containing the agent. As described below, the medical therapy may itself be based on microparticulate (drug delivery) systems, or optionally be independent thereof, with the actual elimination procedure being performed thereafter.

The concept of the present invention is based on the fact that potentially toxic or pathological substances or substances that are harmful to the human or animal body by other means (hereinafter often referred to as toxic and/or harmful substances), e.g. therapeutic agents, can be efficiently eliminated from an organism by means of particles which bind, take up and/or carry said potentially toxic and/or harmful substances, by indirect separation aimed at the particles in an extracorporeal elimination step or optionally in the extrinsic or exogeneous device. A particular advantage of the method according to the present invention is that toxic therapeutic agents can be removed after their peak effect by means of the particulate carrier from a suitable body fluid, in particular from blood, after they were applied for therapeutic purposes in the form of suitable and, in general, known particulate carrier systems. The toxicity of the agent is reduced as a by-effect, whereby the improved tolerability and reduced toxicity of conventional, site-specific and/or target-directed drug delivery systems based on an agent/carrier unit act in combination with the very efficient option of eliminating these macroscopic agent carriers. Moreover, according to the concept of the present invention the toxic substances are removed from the natural clearance cycle and/or physiological metabolism which spares the organs specialized on natural detoxification such as the liver, bile, kidneys, etc.

The method according to the invention can be used to remove from the organism all potentially toxic and/or harmful agents or substances which bind to or are encapsulated by the selected particles or were already bound or encapsulated in the course of a preceding therapy. According to the present invention it is also possible to combine the extracorporeal elimination with the extracorporeal administration of a therapy by means of appropriate body fluids, such as blood. In the extracorporeal cycle, it is possible to adjust for instance the temperature and the pH value to suit the desired biochemical reactions or interactions. A wealth of reference material is available on the properties of biological membranes, e.g. liposome membranes. Concerning temperature—and/or pH-mediated delivery, reference shall be made to two current literature sources, in which several clinical application options are described: A. Hillery: “Heat-sensitive liposomes for tumour targeting”, Drug Discov Today 6(5) (2001), p. 224-225; and I. M. Hafez et al.: “Tunable pH-sensitive liposomes composed of mixtures of cationic and anionic lipids”, Biophys. J. 79(3) (2000), p. 1438-46. With regard to loading commercially available liposomes for therapeutic applications with the agents, for instance a pH gradient can be used to induce the uptake of the agent into the preformed particles (see S. H. Hwang et al.: “High entrapment of insulin and bovine serum albumin into neutral and positively-charged liposomes by the remote loading method”, Chem Pharm Bull (Tokyo) 48(3) (2000), p. 325-9; S. H. Hwang et al.: “Remote loading of diclofenac, insulin and fluorescein isothiocyanate labeled insulin into liposomes by pH and acetate gradient methods”, Int J Pharm 179(1) (1999), p. 85-95; E. Maurer-Spurej et al.: “Factors influencing uptake and retention of amino-containing drugs in large unilamellar vesicles exhibiting transmembrane pH gradients”, Biochim Biophys Acta 1416(1-2) (1999), p. 1-10; D. B. Fenske et al.: “Ionophore-mediated uptake of ciprofloxacin and vincristine into large unilamellar vesicles exhibiting transmembrane ion gradients”, Biochim Biophys Acta 1414(1-2) (1998), p. 188-204; M. Gulati et al.: “Study of azathioprine encapsulation into liposomes”, J. Microencapsul. 15(4) (1998), p. 485-94). This principle can be used not only for loading, but also for elimination or reduction according to the present invention of endogenous metabolic products or exogenous noxious substances (e.g. intoxication) both intracorporeal (in the extrinsic or exogeneous device) and, especially, extracorporeal. For instance, a liposome suspension can be added in vivo as well as ex vivo to a body fluid, e.g. plasma, in order to bind the undesired substances by means of a specific or unspecific transport into the liposomes. In this context, please refer to U.S. Pat. Nos. 5,843,474, 6,079,416, 5,858,400 (K. J. Williams) and U.S. Pat. No. 6,139,871 (Hope and Rodrigueza). The liposomes are eliminated in a subsequent step using one of the methods described below.

The toxic or harmful substances comprise especially therapeutic agents, e.g. conventional pharmaceutical or recombinant agents, any type of DNAs and RNAs as suited for use in gene therapy or antisense technology, radionuclides, etc., as well as other substances that are harmful to the organism. The latter category includes not only exogenous toxic substances or noxious substances, but also endogenous substances whose content or concentration in the body is to be reduced, e.g. because the level of the substance in the body exceeds its normal range. The removal of the substance desired according to the present invention may equally well correspond to all but complete elimination or a desired reduction of the level of the substance. Partial reduction of the substance may be appropriate e.g. if the goal is to adjust the dose of a therapeutic agent in the body or adapt it to the desired pharmacological time course. The type of particle suited for the substance to be removed can be selected without difficulty by an expert in this field, possibly from several available options of types of particles, as shall be described in more detail in the following.

Suitable particles comprise any type of microparticulate carrier or transport vehicles which bind, take up and/or carry the potentially toxic and/or harmful substances and, in particular, the therapeutic agents. Preferably, the particles, which are used according to the invention and are applied to the body prior to the actual elimination step, should show high binding or carrying capacity for the toxic substance in question, be inherently non-toxic or show only limited toxicity, be non-immunogenic, and allow selective supply of the agent during the preceding therapy, if desired. The macroscopic particle carrier may be natural or artificial in origin or an artificial modification of natural vehicles. As an example, microparticulate carrier particles known from conventional drug delivery systems may be used. Liposomes, microspheres, nanoparticles, niosomes, polymer particles, lipoproteins, virus particles (viruses, virus capsids, and other, modified virus particles, whose virulence was removed or otherwise modified), and certain cell types, such as subtypes of blood cells, e.g. erythrocytes and lymphocytes, are particularly well-suited for this purpose.

Microparticulate agent carrier systems of this type have been described, e.g. by P. Zanoviak, “Pharmaceutical Dosage Forms”, in particular in Chapter 7.3, in “Ullmann's Encyclopedia of Industrial Chemistry”, Vol. A 19, 5^{th} ed., 1991, p. 241-271, and in the following other sources: E. Timlinson: “Site-Specific Drug Delivery” in G. S. Banker, C. T. Rhodes (eds.): Modern Pharmaceuticals, 2^{nd} edition, Marcel Dekker, New York 1990, p. 673-694; S. N. Mills, S. S. Davis: “The Targeting of Drugs/Controlled Drug Delivery” in L. Illum, S. S. Davis (eds.): Polymers in Controlled Drug Delivery, IOP Publishing, Bristol 1987, p. 4-6; P. Arthurson: “Site Specific Drug Delivery/The Fate of Microparticulate Drug Carriers after Intravenous Administration” in L. Illum, S. S. Davis (eds.): Polymers in Controlled Drug Delivery, IOP Publishing, Bristol 1987, p. 15-24; R. L. Juliano, D. Layton: “Liposomes as a Drug Delivery System” in R. L. Juliano (ed.): Drug Delivery Systems, Oxford University Press, New York 1980, p. 189-236; and R. C. Oppenheim: “Nanoparticles” in R. L. Juliano (ed.): Drug Delivery Systems, Oxford University Press, New York 1980, p. 177-188. Other suitable agent/carrier conjugates have been described in: J. P. Benoit et al.: “Les formes “vectorisées” ou a “distribution module”, nouveaux systèmes d'administration des médicaments”, J. Pharm. Belg. 41 (1986): p. 819-829; F. Emmen and G. Storm: “Liposomes in Treatment of Infectious Diseases”, Pharm. Weekblad (Sci) 9 (1987): p. 162-171; G. Gregoriadis, J. Senior, and A. Trouet (eds.): “Targeting of Drugs”, Plenum Press, New York 1982; G. A. Kruse et al.: “Mouse Erythrocyte Carriers Osmotically Loaded with Methotrexate”, Biotechnol. Appl. Biochem. 9 (1987): p. 123-140; R. Lawaczeck: “Liposomen als Zielgerichtete Pharmakaträger”, Deutsche Apotheker-Zeitung 127 (1987): p. 1771-1773; and U. Sprandel and R. A. Chalmers: “Morphologie von Erythrozytenschatten als in-vivo-Trägersysteme” in: Verhandlungen der Deutschen Gesellschaft für Innere Medizin, 86. Congress, Wiesbaden (Germany) 1980 (B. Schlegel, Ed.), Bergmann Verlag, Munich 1980; with regard to radionuclide loading please refer to: K. Kostarelos and S. Emfietzoglou: “Tissue dosimetry of liposome-radionuclide complexes for internal radiotherapy”, Anticancer Res. 20 (5A) (2000), p. 3339-3345).

The particles employed for this use may themselves contain agents or auxiliaries, or may be used as and supplied to the organism as empty vesicles or particles which can bind or receive toxic or harmful substances in order to take up the substances that are undesirable in the body, whereby, subsequent to the loading within the organism, the loaded particles are eliminated from the organism. Therefore, the method according to the procedure is particularly favorable to use, without being limited to this case, if the particles to be removed serve as agent carriers in a preceding medical therapy. Rather, as an option, the particles identified above can be used such that they are applied to the body as such, i.e. without any agent being bound or loaded. Under these circumstances, the carrier materials identified above can bind or receive under in vivo conditions the toxic and/or harmful substances that are present in the body and need to be removed. After this follows a subsequent—preferably extracorporeal—elimination step for removal of the particles which now carry toxic substances. Especially in this application, efficient control in terms of optimal control over the dose and the time course of application of the substance is a great advantage. If the potentially toxic substances or therapeutic agents entered the organism in their free form or by means of carrier particles, any excess of the agents that is not bound to cellular structures or organs of the organism can be eliminated or at least reduced in concentration. Thus, it becomes clear that the present invention is not limited to therapeutic agents, but may also be used to eliminate or detoxify other potentially toxic substances from or in the body. Accordingly, the present invention can be used to eliminate not only endogenous harmful and/or undesired substances, but also poisoning and similar intoxications. Moreover, the option of complete or partial elimination by means of removing particles which bind the toxic or harmful substances may also be advantageous for control or adjustment of the levels of the corresponding substance within the organism or for improvement of the dosage or the time course of a therapeutic regimen utilizing a therapeutic agent.

As a function of the composition of the lipids or proteins and apolipoprotein composition, the particles show different binding affinities, half-life values, chemical binding properties and electrostatic interactions with body tissues or cells including both healthy cells and cells after malignant transformation (receptor composition). The specificity of binding, internalization, degradation, and ensuing (intra-) or extracellular release and effect in or on the cells can be increased by varying the composition of the lipoprotein particles (natural or artificial), liposomal particles, and carrier systems identified above. The so-called clearance (uptake into cells or elimination by means of body fluids, such as bile, urine, etc.) and metabolization (e.g. degradation in liver and/or kidney) of the particles and transported substances can be influenced accordingly or modified with the present invention.

Receptor-mediated drug targeting may be mentioned as an example, in which differences in receptor or surface proteins or markers (e.g. glycoproteins, “glycocalix”) composition lead to differences in the affinity for agent-carrying or “empty” particles.

The transfer to other body fluids (e.g. from blood to urine) may accumulate the particles or agent. This enrichment may be desirable from a therapeutic point of view.

With regard to both the premade therapeutic form and the in vivo binding for uptake of toxic or harmful substances, the substance or substances may bind to the particles by any suitable means, with chemical bonds, such as covalent bonds, electrostatic interactions, hydrophobic or hydrophilic interactions, specific conjugate formation by means of antibodies or antibody fragments or receptor binding, incorporation in a particle membrane or its aqueous or lipophilic phase as well as pure physical absorption or adsorption being suitable means.

Examples of empty particles according to the present invention which are suitable to take up toxic or otherwise harmful substances include the liposome systems described by K. J. Williams in U.S. Pat. No. 5,843,474, U.S. Pat. No. 6,079,416, and U.S. Pat. No. 5,858,400 and by M. Hope and W. Rodrigueza in U.S. Pat. No. 6,139,871. However, the systems referred to above are based on a different approach: they concern therapeutic approaches as systemic means to contribute to a reduction of LDL, cholesterol, or plaque levels as a treatment for atherosclerosis or renal disease without considering elimination by means of removing from a body fluid the particles loaded with the harmful substance in an extracorporeal step or in a device outside the body. Please refer also to the following related references: K. J. Williams et al.: “Structural and metabolic consequences of liposome-lipoprotein interactions”, Adv. Drug Deliv. Rev. 32 (1-2) (1998), p. 31-43; K. J. Williams et al.: “Rapid restoration of normal endothelial functions in genetically hyperlipidemic mice by a synthetic mediator of reverse lipid transport”, Arterioscler. Thromb. Vasc. Biol. 20 (4) (2000), p. 1033-1039; M. Aviram et al.: “Macrophage cholesterol removal by triglyceride-phospholipid emulsions”, Biochem. Biophys. Res. Commun. 155 (2) (1988), p. 709-713. On the other hand, according to the present invention, the conjugates formed in or on the particles after uptake or binding of the toxic or harmful substance are eliminated in the subsequent elimination step. Artificial lipoproteins, as described by Barenholz et al. (U.S. Pat. No. 5,948,756) and Levine et al. (U.S. Pat. No. 5,128,318) can also be used for in vivo uptake of toxic or harmful substances and are subsequently removable according to the present invention.

Moreover, it is known that erythrocytes can be used to take up and encapsulate agents under in vivo conditions. The dissertation of Dr. Klaus Clauβen (dissertation of the Fakultät für Naturwissenschaften of Martin-Luther-Universität Halle-Wittenberg, August 1989) demonstrates that erythrocytes can be loaded with selected estrogens under in vivo conditions.

As shown above, loading under in vivo conditions prior to elimination can be achieved in particular by means of chemical affinities, such as electrostatic interactions or hydrophobic interactions, or by means of the formation of specific conjugates, e.g. by means of specific antibodies against the toxic substance to be removed, said antibodies being bound to the particles, or through the use of other auxiliary substances such as receptors or acceptors. For this purpose, the composition of the natural, artificial or modified particles listed above may be varied and auxiliary substances aiding the binding step may be added depending on the nature of the substance to be bound or taken up.

Liposomes are particularly preferred amongst the microparticles specified by the present invention. Liposomes are well-characterized and their properties and compositions are easy to vary for the purposes of the present invention. A plethora of different compositions and structures of liposomes are known and available and ready for use in practical applications for the treatment of diseases with the agent loaded therein. In this context, please refer to D. D. Lasic: “Novel applications of liposomes”, Trends Biotechnol. 16(7) (1998), p. 307-321; A. Chonn and P. R. Cullis. “Recent advances in liposomal drug-delivery systems”, Curr Opin Biotechnol. 6(6) (1995), p. 698-708; and U. Massing: “Cancer therapy with liposomal formulations of anticancer drugs”, Symposium Pharmacokinetics and Oncology, p. 87, based on a congress of the Society of Clinical Pharmacology and Therapy, Cologne, Oct. 17, 1996. According to the invention, it was discovered that this type of particle allows very efficient elimination of the toxic or harmful substance-loaded liposomes in the subsequent elimination step.

Hitherto, lipoproteins with a rather low density, i.e. the so-called “low density lipoproteins” (LDL) have been preferred. The LDL lipoprotein is known to easily bind or take up low molecular weight substances, and this includes toxic substances, under in vivo conditions. Moreover, there are well-established apheresis methods for the selective removal of the LDL lipoprotein fraction in extracorporeal steps. However, by modifying hitherto conventional LDL apheresis methods or, as an option, by means of other elimination procedures directed at the respective target substances it is possible to use and subsequently eliminate not only LDL carrier particles, but also lipoprotein particles with different densities, such as HDL, IDL, VLDL, so-called beta-VLDL, chylomicrons and chylomicron remnants. Suitable drug delivery systems based on lipoproteins of varying lipid composition and density are described for example in T. J. C. van Berkel et al.: “Drug targeting by endogenous transport vehicles”, Biochem. Soc. Trans. 18(5) (1990), p. 748-750; H. W. Schulties et al.: “Preparation of nucleoside-LDL-conjugates for the study of cell-selective internalization stability characteristics and receptor affinity”, Eur. J. Clin. Chem. Clin. Biochem. 29 (1991), p. 665-674; J. Mankertz et al.: “Low density lipoproteins as drug carriers in the therapy of macrophage-associated diseases”, Biochem. Biophys. Res. Commun. 240 (1997), p. 112-115; H. W. Schulties et al. “Functional characteristics of LDL particles derived from various LDL-apheresis techniques regarding LDL-drug-complex generation”, J. Lipid Res. 31 (12) (1990), p. 2277-2284; and P. C. de Smidt and T. J. van Berkel: “LDL-mediated drug targeting”, Crit. Rev. Ther. Drug Carrier Syst. 7(2) (1990), p. 99-120.

The present invention also considers the elimination of viruses or virus-like particles. By means of the techniques described in more detail below and by further development of these techniques, viruses present in body fluids during viral infections can be bound and eliminated. Due to its chemical-biological composition, the use of the viral capsid, which is used in a medical development for instance as an attenuated viral vaccine, is considered by the present invention as a means for the transport and application of any associated agents as well as for their elimination. In this context, reference shall be made to the application and subsequent desired (and in some cases—partial) elimination of substances used in the so-called “gene therapies”.

The size of the particles to be used according to the invention may vary depending on the type of particle and usually is on the scale of nanometers to micrometers. To provide for easy passage through the vascular system, the mean particle size preferably is in the range from 10 nm to 10 μm. With regard to particles of artificial origin, such as nanospheres, nanoparticles, polymer particles, artificial or natural, possibly artificially-modified lipoproteins and especially liposomes, mean sizes with an average diameter of 10 to 300 nm, in particular 40 to 200 nm are preferred, because this range provides a good compromise between the tendency of relatively large particles to be phagocytosed and the loading capacity of the particles. In contrast, if particles of natural origin are used, larger particle sizes corresponding to their natural size are more relevant, especially in the use of certain types of cells, such as erythrocytes or lymphocytes, or certain lipoprotein particles.

According to the present invention, the particles described above which carry or are loaded with the potentially toxic substances, such as therapeutic agents or noxious substances, are eliminated from a body fluid in an extracorporeal step or in an exogeneous or extrinsic device subsequent to their application to the body. In the former alternative, i.e. the extracorporeal step, a suitable body fluid comprises a body or tissue fluid previously withdrawn from the body, in particular blood or blood plasma. In the alternative case, the exogeneous or extrinsic device may be a device for removal of particles from a fluid present in a body cavity or a tissue fluid, such as from ascites, pleural effusion, urine, from the peritoneum, secretions or eliminated fluids, such as saliva, liquor, bile, lymph, pancreas secretion, etc.

The extracorporeal removal of the particles is best done by removing the blood with selective blood detoxification procedures. Blood detoxification procedures of this type are generally known and familiar to the expert and these procedures can be adapted to suit the particles to be removed depending on the type and characteristics of the particles. Compared to conventional blood detoxification procedures for the removal of toxic or pathogenic substances from blood, the method according to the invention on the basis of carrier particles provides clear advantages, since toxic substances bound to particle carriers can be removed from the blood more specifically and more efficiently. Moreover, if there is a preceding attempt at therapy, the invention forms a combination with the preceding therapeutic approach on the basis of drug delivery systems based on particle carriers. Thus, the present invention facilitates the efficient removal of the particle-bound toxic substance or agent from the blood after its peak effect is achieved without any further systemic toxicity and by sparing the body's inherent clearance system and associated cell types and organs (phagocytosis system, liver, kidney, and spleen), whereby the elimination is achieved by means of procedures that are excellently established and in clinical use at many medical centers. For a description of suitable procedures in the literature, please refer to B. R. Gordon and S. D. Saal: “Current status of low density lipoprotein-apheresis for the therapy of severe hyperlipidemia”, Curr. Opin. Lipidol. 7 (1996), p. 381-384; K. Kajinami and H. Mabuchi: “Therapeutic effects of LDL apheresis in the prevention of atherosclerosis”, Curr. Opin. Lipidol. 10 (1999), p. 401-406; N. Koga: “Efficacy and safety measures for low density lipoprotein apheresis treatment using dextran sulfate cellulose columns”, Ther. Apher. 3 (1999), p. 155-160; R. S. Lees et al.: “Non-pharmacological lowering of low density lipoprotein by apheresis and surgical techniques”, Curr. Opin. Lipidol. 10 (1999), p. 575-579; G. R. Thompson and Y. Kitano: “The role of low density lipoprotein apheresis in the treatment of familial hypercholesterolemia”, Ther. Apher. 1 (1997), p. 13-16; T. Yamamoto and T. Yamashita: “Low-density lipoprotein apheresis using the Liposorber system: features of the system and clinical benefits”, Ther. Apher. 2 (1998), p. 25-30. The method according to the present invention even facilitates the recovery of the agent-containing or agent-carrying particles after the removal of these particles from the body fluid, and reuse thereof in the therapeutic application, whereby the required sterility and non-objectionability of the recovered and re-applied drug delivery particles in terms of their microbiological and infection properties must be guaranteed.

Depending on the size and type of the particles to be eliminated from a body fluid, processes based on the precipitation, filtration, chromatography and/or adsorption of the particles are particularly suited for eliminating said particles. According to the invention, it has been found that for instance conventional blood apheresis procedures can be used for this purpose. Apheresis procedures of this type are known for instance for the selective reduction of the LDL content of blood in the treatment of hypercholesterolemia.

These time-proven apheresis procedures can be applied very well and very easily for the extracorporeal removal of the particles loaded with the toxic substances or agents. Various therapeutic apheresis procedures are applied in ten thousandths of cases in Germany alone each year and, except for some variation between the individual methods, are generally tolerated very well by the patients. This includes the so-called HELP system (see EP 0 174 478 A2; commercially exploited by B. Braun, Melsungen, Germany), double-filtration technique (cascade filtration) or membrane differential filtration (MDF), DALI procedure for direct adsorption from whole blood (see K. Derfler and W. Drumel in Eur. J. Clin. Invest. 28 (1998): 1003-1005; L. J. Dräger et al. in Eur. J. Clin. Invest. 28 (1998): 994-1002; commercially exploited by Fresenius A G, St. Wendel, Germany), adsorption from blood plasma on dextran sulfate (see N. Koga: “Efficacy and safety measures for low density lipoprotein apheresis treatment using dextran sulfate cellulose columns”, Ther. Apher. 3 (1999), p. 155-160; and T. Yamamoto and T. Yamashita: “Low density lipoprotein apheresis using the Liposorber system: features of the system and clinical benefits”, Ther. Apher. 2 (1998), p. 25-30; commercially available under the name, LIPOSORBER™, from Kaneka, Osaka, Japan), and immunoadsorption. The cited references as well as Wiener Klinische Wochenschrift, vol. 112(2) (2000), see editorial on p. 49-51, present an overview over the various apheresis procedures for the treatment of hypercholesterolemia. If the particles are not removed directly from whole blood, the blood cells must first be separated, e.g. by filtration or centrifugation and subsequent removal of the blood cells from the plasma.

Although it is easiest to remove the particles from blood or blood plasma using existing procedures, the particles can also be removed from any other body fluid after withdrawing said fluid from the body, e.g. tissue fluid, ascites, pleural effusion, urine, fluid from the peritoneum, secretions or eliminated fluids, such as saliva, liquor, bile, lymph, pancreatic juice. If desired for physiological or therapeutic reasons, the body fluid can then be returned to the body after the particles are removed.

In order to remove the particles by precipitation it is preferably to use a polyvalent charged or ionizable precipitating agent, such as polyanions or polycations, in combination with oppositely charged or ionizable particles or particle fragments. Preferred examples of polyanions as precipitating agents for positively charged or ionizable particles include heparin, dextran sulfate, and phosphotungstic acid. Suitable alternatives include other polyvalent polymers, such as polysulfate, polysulfonate, polyphosphate, polycarboxylate, poly(meth)acrylate, polyvinylsulfate, polyvinylsulfonate and polyvinylphosphate, polystyrenecarboxylate and polystyrenesulfonate, anionic polysaccharides, such as polygalacturonates, hyaluronic acid, keratin sulfate, anionic cellulose derivatives, algininic acid, etc., anionic polypeptides, such as polymers or copolymers with glutamic acid units and/or aspartic acid units, copolymers of the units of the polymers named above, and similar substances. Suitable polycations for use as precipitating agents for negatively charged or ionizable particles include alkaline polysaccharides, such as dextrans, amylose, amylopectin or other polysaccharides with primary, secondary or tertiary amino groups or alkylammonium groups, polypeptides or their copolymers with alkaline amino acid units, such as lysine, arginine, ornithine, and polymeric amines or N-heterocyclic substances, such as polyvinylamines, polyallylamines, polyvinylalkylamines, and polyvinyltrialkylammonium salts, polyvinylpyridines and their quaternary salts, poly(aminoalkyl)vinyl alcohols, copolymers of the units of the polymers named above and similar substances.

The precipitation is suitably conducted in the presence of divalent cations, such as Ca²⁺, Mn²⁺ and/or Mg²⁺. The precipitating agents and possible auxiliary substances may be added after plasma separation as exogenous substances, e.g. in the form of suitable solutions or buffer mixtures. The original concentrations can be reestablished through the use of a suitable downstream device, such as a dialysis unit, adsorption column, etc. Similarly, any undesired substance that were added to the reaction mixture can be removed prior to the return to the patient.

The use of polyanions as precipitating agent is sensible if the particles carry positively charged or ionizable groups, such as primary, secondary or tertiary amines or quaternary ammonium groups, as is the case. for instance, in the use of lipoprotein particles because of the ApoB component of LDL lipoprotein, and in the use of positively charged or chargeable liposomal membrane components. There is a plethora of positively chargeable or permanently positively charged liposomes available for use for the purposes of the present invention. In this context, please refer to the following references: O. Zelphati and F. C. Szoka, “Mechanism of oligonucleotide release from cationic lipids”, Proc. Natl. Acad. Sci. USA 93 (21) (1996), p. 11493-11498. The known and commercially available cationic lipids described in U.S. Pat. No. 6,020,526 as liposome components include N[1-(2,3-dioleoyloxy)propyl]-N,N,N-trimethylammoniumchloride (“DOTMA”), dioleoylphosphatidylethanolamine (“DOPE”), 1,2-bis(oleoyloxy)-3,3,3-(trimethylammonia)propane (“DOTAP”) and structural variations thereof, both alone or in combination, as well as the cationic lipids on the basis of amides which are proposed in this document. Other cationic lipid compounds that have been described include those, in which for instance carboxyspermine was conjugated with one or several lipids, e.g. 5-carboxyspermylglycine-dioctaoleoylamide (“DOGS”) and dipalmitoyl-phosphatidylethanolamine 5-carboxyspermylamide (“IDPPES”); please refer for instance to Behr et al., U.S. Pat. No. 5,171,678. Moreover, cationic cholesterol derivatives (“DC-Chol”) jointly incorporated into liposomes with DOPE have been described as lipid components (see Gao, X. and Huang, L., Biochim. Biophys. Res. Commun. 179 (1991), pp. 280). Moreover, there is lipopolylysine which is produced by conjugating polylysine to DOPE (see Zhou, X. et al., Biochim. Biophys. Acta 1065 (1991), pp. 8). Other suitable lipid components for use in cationic liposomes are described in U.S. Pat. No. 6,172,049.

On the other hand, there is the option to use anionic lipid components to build up anionic liposomes that can be precipitated with cationic precipitating agents, e.g. with the anionic lipids, phosphatidic acid, phosphatidylglycerol, phosphatidylglycerol-fatty acid ester, phosphatidylethanolamine and amide, phosphatidylethanolamine and amide, phosphatidylserine, phosphatidylinositol, phosphatidylinositol-fatty acid ester, cardiolipin, phosphatidylethyleneglycol, acidic lysolipid, sulfolipid, sulfatide, saturated or unsaturated free fatty acids, such as palmitic acid, stearic acid, arachidonic acid, oleic acid, linolenic acid, linolic acid, and myristic acid, which are described in U.S. Pat. No. 6,120,751.

Other charged or ionizable lipids components for building up the correspondingly charged liposomes are commercially available from Avanti Polar Lipids, Inc., Alabaster, Ala. 35007, USA (www.avantilipids.com).

Aside from the lipid components, other components and auxiliary substances can be incorporated into the liposomes. This includes for instance the substances for specific binding mentioned above (for targeted therapy and/or specific elimination), liposome stabilizers, such as polyethyleneglycol (PEG) or other functional components and polymers. These components, which are not lipid components, can for instance support the precipitation or other elimination techniques. Polymer components other than PEG include e.g. polyvinylpyrrolidone, polyvinylalcohol, polypropyleneglycol, polyvinylalkylether, polyacrylamide, polyalkyloxazoline, polyhydroxyalkyloxazoline, polyphosphazine, polyoxazolidine, polyaspartic acid amide, a polymer of sialic acid, polyhydroxyalkyl(meth)acrylate, and poly(hydroxyalkylcarbonic acid).

It has been found that the common conditions and steps of the conventional HELP procedure for eliminating LDL particles (heparin in divalent cation-containing acidic buffers with a pH in the range of 4.8-5.4) are applicable not only to the heparin-induced precipitation of LDL, but also to liposomes containing positively charged or ionizable groups, such as phosphogycerides with phosphatidylcholine and phosphatidylethanolamine units.

If it is intended to remove the particles based on a filtration process, the technique of membrane differential filtration (MDF) or cascade filtration proves to be well-suited. This techniques affords a more efficient elimination of the particles employed because it applies at least two filter systems subsequent to the preceding separation of the blood cells and plasma and followed by subsequent filtration-based steps separating the microparticulate components employed in the procedure from other plasma components. The individual filter systems can be adjusted or selected in terms of the pore size of their filter materials to suit the size of the individual blood components and especially the size of the particles to be eliminated.

Various adsorption procedures also provide for efficient elimination of the particles and shall be described in more detail in the following.

One option based on the adsorption technique involves that the adsorption is mediated by electrostatic interactions between the particles containing charged groups or being ionizable, and the adsorbent material carrying the corresponding opposite charge or being oppositely ionizable. Suitable materials for this adsorption principle include polycationic or polyanionic adsorbent materials for eliminating the particles carrying oppositely (negative or positive) charged or ionizable groups. For some examples, please refer to the polyanions or polycations described above in connection with precipitation, in particular polyanions, such as polyacrylamide or dextran sulfate, which are very suited for use with positively charged or chargeable LDL or liposome particles. The polycations or polyanions may be ligands that are covalently bound to the corresponding adsorption carrier materials, such as polyacrylamide, Sephadex™, etc. In this context, reference shall be made to the DALI apheresis procedure mentioned above (for reference see above) because it is particularly well-suited and efficient. Although hitherto known only for treatment of hypercholesterolemia, this procedure can also be applied for eliminating carrier particles, as those specified by the present invention, for toxic substances or agents other than LDL particles, e.g. liposomes. The main advantage of the procedure is that the purification step can be performed on whole blood. The approach of the DALI technique is based on a combination of adsorption and size exclusion chromatography (gel chromatography). For this purpose, the small adsorber beads are provided to be porous and have a mean diameter of 150-200 μm with a coating of polyanions, in particular polyacrylate, adhering to the inner and outer surfaces (of the pores) as adsorbent materials.

Immunoadsorption is a specialized variety of the adsorption technique and provides another option for separating off the particles. Antibodies interacting specifically with components or surface components of the microparticles used according to the invention can be bound by covalent bonds to an adsorbent carrier and thus can be used for selective removal of the microparticles from the body fluid withdrawn from the body or from a follow-up product obtained after several intervening separation steps. This provides an excellent foundation for the implementation of the concepts of target-specific therapy in combination with selective elimination a certain time period after the administration of the corresponding drug delivery systems. The selectivity required for targeted therapy and recognition by the antibodies bound to the adsorbent material is conveniently provided by a definite structural feature (“structural-code”): e.g. a microparticle-bound, carcinoma-specific antibody may provide for the selectivity of the site-specific therapy and at the same time serve as an epitope-carrying antigen for recognition by an counter-antibody bound to an adsorbent material. Another target-directed drug delivery system that is combined with an extracorporeal elimination step are specialized cells, such as cytotoxic T cells and NK cells (natural killer cells), which, following their therapeutic application, can be eliminated from the blood circulation by immunoadsorption by means of their cell-specific antigens. Using a similar approach, the viruses and modified viruses mentioned above can also be used and eliminated.

However, toxic or harmful substances, such as cell types containing therapeutic agent, can be removed from the blood by means of blood separation procedures, e.g. leukopheresis, which are specifically adapted to the cell type at hand.

Chromatographic procedures can also be used as specific removal procedures in cases, in which the size of the microparticles taking up toxic substances is relatively small, i.e. on the scale of nanometers or subnanometers. In these cases, it is sensible to apply preceding gross separation steps, such as plasma separation. The chromatographic approach can be based on separation by size differences, or on electrostatic binding to a solid phase carrier (ion exchange chromatography) and/or on formation of an adsorptive bond to hydrophobic solid phase carriers or on an antigen/antibody interaction with a specific solid phase carrier. For instance, E. Choice et al. (Anal. Biochem. 270 (1999), p. 1-8) and J. Turanek et al. (Anal. Biochem. 218 (1994), p. 352-357) describe the chromatographic preparation of liposomes by FPLC. However, this work does not involve a therapeutic approach and does not in any way envision deliberate or substantial elimination of liposomes loaded with toxic substances. Rather, Choice et al. describe the FPLC method as an analytical option for investigating the stability of liposomes in blood and characterizing liposomal drug delivery systems, whereas the reference of J. Turanek applies the FPLC method to the preparation liposomes by the extrusion technique.

It is to be noted that the procedures or steps for eliminating the particles from the body fluid after withdrawal of the fluid from the body or in an exogenous or extrinsic device, as described above, can be conducted alone or in combination or in combination with other, conventional blood detoxification procedures. The elimination according to the invention is suitably conducted in a cycle, in which the elimination step is conducted between the withdrawal of the body fluid and the return of the body fluid to the body after the elimination step. Consequently, components of the body fluid can be returned into the body after the elimination step, in as far as this is desired. A procedure of this type is easy to incorporate into the well-established dialysis or apheresis procedures.

If the present invention is applied after a preceding therapy with microparticulate drug delivery systems, any therapeutic agents are suitable for binding to or inclusion in the particles as a matter of principle. However, the application of the elimination system according to the invention provides for the development of a novel pharmacology for known agents. This is of greatest interest especially in those areas, in which highly toxic, very harmful or for the organism critical substances are employed. In this context, the procedure according to the invention combined with a preceding medical therapy is particularly sensible, if the therapeutic agents are selected from the group consisting of cytostatic agents, antibiotics, antivirals, antimycotic agents, gene-therapeutic substances (i.e. all substances used in so-called gene therapies, such as oligonucleotides, ribozymes, DNAs, plasmids, vectors, and liposome- or virus particle-based gene shuttles), antibodies, cytokines, interferons, and radionuclides. The cytostatic agents include e.g. the agents, daunorubicin and doxorubicin, which are already provided in liposomal form, as well as N-lost derivatives, ethyleneimines, alkylsulfonates, nitrosourea derivatives, platinum complex compounds, dacarbazine and procarbazine, mitomycin, altretamine, intercalating cytostatic agents, such as actinomycins, anthracyclins, mithramycin, amsacrine, mitoxantrone, antimetabolites, such as folic acid antagonists, pyrimidine antagonists, purine antagonists, spindle toxins, such as vinca alkaloids and podophyllotoxin derivatives, as well as bleomycins, hydroxyurea, mopidamole, L-asparaginase, and interferons, etc. The series of these and other classes of agents is unlimited. Aside from the general literature references cited above, special reference shall be made to the articles by D. D. Lasic (1998) (see above) and A. Chonn and P. R. Cullis (1995) (see above) with regard to microparticulate drug delivery, and to the article by K. Kostarelos and D. Emfietzoglou with regard to liposome-mediated radiotherapy (see above). The combination of a therapeutic approach and (possibly partial) subsequent elimination of the therapeutic agent is also sensible to use in the problem-ridden approaches of gene therapy, in which oligonucleotides, such as antisense agents, ribozymes, DNA plasmids, vectors or similar gene shuttles based on viruses or liposomes are employed; for examples, please refer to E. M. Hersh and A. T. Stopeck: “Advances in the biological therapy and gene therapy of malignant disease”, Clin. Cancer Res. 3 (1997), p. 2623-2629; R. R: Weichselbaum and D. Kufe: “Gene therapy of cancer”, Lancet 349 Suppl. 2 (1997), SII10-SII12, P. J. Woll and I. R. Hart: “Gene therapy for lung cancer”, Ann. Oncol. 6 Suppl. 1 (1995), 73-77; and J. M. Brown and A. J. Giaccia: “The unique physiology of solid tumors: opportunities (and problems) for cancer therapy”, Cancer Res. 58 (7) (1998), p. 1408-16.

The agents may be synthetic, natural or semisynthetic in origin or may have been produced by microbiological or gene technology means. The type and production of microparticulate drug delivery systems containing this type of agent have been described in the literature.

The present invention also provides a kit for medical therapy or experimental or research purposes, said kit comprising

a) a preparation of particles which can bind, take up and/or carry potentially toxic or harmful substances, and

b) means for the removal of the particles from body fluids.

With regard to this kit and referring to the particles which bind, take up or carry the potentially toxic or harmful substances, reference shall be made to the description above, in particular to the selection and different designs of liposomes, selectively removable cell types, lipoproteins, and polymer particles. The kit is particularly useful, if the toxic or harmful substances comprise therapeutic agents, endogenously formed or exogenous-added potential noxious substances. The particles provided in the kit may already encapsulate the therapeutic agents provided sufficient stability is ensured. Optionally, the particle samples and the sample containing the therapeutic agent may be provided as separate components of the kit for combination prior to use in order to load the agent in/on the particles. An example for the latter case is the therapeutic combination consisting of liposomes and doxorubicin which is commercially available from TLC under the name, “TLC D-99”. The user of this product loads the agent in/on the liposomes only directly before the injection.

With regard to the therapeutic agents, reference shall also be made to the illustrations provided above.

Which means for removing the particles from body fluids, such as blood, blood plasma, tissue fluids, such as ascites or pleural effusion, secretions or eliminated fluids, such as saliva, liquor, bile, lymph, pancreatic juice, urine, peritoneal fluid, etc., to use is determined by the selected purification procedures described above in that these means correspond to the technical means that are suitable or common for the conduct of the respective procedure. Thus, the means provided in the kit include suitable precipitation means, filtration means, adsorption means and/or chromatography means as well as the corresponding required or common reagents, such as buffers, washing solutions or other auxiliary substances, both alone and in combination.

Because of the fact that liposomes are quite common in practical applications and a wide variety of liposome types with varying characteristics is available, another aspect of the invention provides for the use of such liposomes for eliminating toxic substances from the human body. In a very useful application thereof, a therapeutic agent is the toxic substance and the liposomes loaded with this agent are used for treatment of diseases prior to the elimination of the agent from the human body in order to prevent its toxic effects. Usually, a therapeutic approach of this type involves the application of the agent-loaded liposomes by conventional means, i.e. currently by means of an intravenous route, in the future possibly by oral application or through the use of an alternative application route, followed by at least partial removal of the liposomes from the body after a time period selected according to desire or need. The liposomes can be removed using any of the elimination procedures described above.

Therefore, the present invention provides very efficient means for removing agents from body fluids, such as blood. By means of the microparticles used according to the invention, the therapeutic agents can be removed with relatively little effort after they reach their peak effect, especially in the case of carrier-bound drug delivery systems, by means of procedures for the removal of particulate components from body fluids, such as blood and blood plasma. Conventional and well-established blood washing/blood detoxification or apheresis procedures and conventional particulate agent carrier conjugates can be used for this purpose. The application of the approach according to the invention facilitates the use even of highly toxic agents which used to be contraindicated or indicated only in extreme situations due to their toxic side effects. Furthermore, the system according to the invention provides the basis for a novel pharmacology for known agents: e.g. after the application of an agent at high doses and ensuing accumulation of the agent/particle conjugates at the desired target site, e.g. a tumor, the dose level present in the blood can be reduced again by application of the system of the invention. Combined with a modified pharmacology characterized by reduced toxicity, the use of the approach according to the invention thus promises to provide for higher efficacy and a lower risk of inducing the organism's inherent resistance against the agents (multi-drug resistance). The organism is spared because of the application of the present invention in that the detoxified body fluids, especially as the separated blood components, can be returned to the body in a cycle such as is common in conventional blood washing or apheresis procedures. All publications cited in this application are herewith included in the present disclosure.

In the following, the present invention is illustrated on the basis of several non-limiting examples.

### EXAMPLES

The following abbreviations are used:

| | |
|---|---|
| DPPC | 1,2-O-dipalmitoyl-sn-glycero-3-phosphocholine |
| DPDAP | 1,2-O-dipalmitoyl-3-dimethylammonium-propane |
| DPTAP | 1,2-O-dipalmitoyl-3-trimethylammonium-propane |
| DSPE | 1,2-O-distearoyl-sn-glycero-3-phosphoethanolamine |
| DSPG | 1,2-O-distearoyl-sn-glycero-3-phospho-rac-(1-glycerol) |
| EGTA | [bis-(aminoethyl)-glycolether]-N,N,N′,N′- |
| | tetraacetic acid, disodium salt |
| HEPES | 2-[4-(2-hydroxyethyl)-1-piperazinyl-]- |
| | propanesulfonic acid |
| PC | Phosphatidylcholine |
| POPC | 1-O-palmitoyl-2-O-oleoyl-sn-glycero-1-phosphocholine |
| rpm | revolutions per minute |
| v/v | volume per volume |

1. General Description of Materials and Methods
1.1 Radioactive substances
[1,2-³H]cholesterylhexadecylether (specific radioactivity: 1.9 TBq/mmol) supplied by NEN Life Science (Frankfurt, Germany).
1.2 Non-Radioactive Substances

The following companies supplied the chemicals listed:

Avanti Polar Lipids, Inc. (Alabaster, Ala., USA): DPTAP, DPDAP

Pharmacia LKB Biotechnology (Uppsala, Sweden): Heparin-Sepharose

Sigma-Aldrich Chemie GmbH (Deisenhofen, Germany): cholesterol Sygena Ltd. (Liestal, Switzerland): DSPE, DPPC, DSPG, POPC Zinsser Analytik GmbH (Frankfurt, Germany): Biolute-S®

All other chemicals were procured from the common manufacturers at the highest purity offered.

1.3 Preparation of Buffer Solutions

The buffer solutions were prepared using water from a Milli-Q UF water purification system (Millipore GmbH, Eschborn, Germany) with a resistance of at least 19 MW·cm.

1.4 UV/VIS Spectroscopy

A UV/VIS spectrometer (DU 600, Beckman Instruments, Inc., Fullerton, USA) was used for the spectroscopic measurements at UV and visible wavelengths. Depending on the experimental design, 0.5 or 1 cm quartz cuvettes were used.

1.5 Measurement of Radioactivity

The radioactivity in liquid samples was determined by liquid scintillation measurement (Wallac 1411, Berthold, Wildbad, Germany). Quench correction and an external standard were used for absolute radioactivity measurements. For measuring the radioactivity in samples, 10 ml Ultima Gold® (Packard) was added to maximally 1 ml of the sample solution. The radioactivity was determined no earlier than after 4 hours.

### 2. Preparation and Characterization of Liposomes

The liposomes were prepared according to a modified version of the extrusion method of MacDonald et al.: “Small-volume extrusion apparatus for preparation of large, unilamellar vesicles”, Biochim. Biophys. Acta 1061 (1991), p. 297-303. The individual lipid components of the liposome membrane to be prepared were dissolved at defined concentration in chloroform (POPC, DPPC, cholesterol) or a mixture of chloroform and methanol (2/1, v/v) (DSPG, DSPE, DPDAP, DPTAP). These stock solutions were stored at 20° C. In order to prepare liposomes with a desired lipid composition, the corresponding aliquots of the lipid stock solutions were transferred to a 25 ml round-bottom flask. The lipid composition of the liposomes used in the experiments is shown in Table 1 (in units of mol %).

TABLE 1
Lipid composition of the liposomes used
in the experiments (in units of mol %)

| | DSPG- | | DSPE- | DPDAP- | DPTAP- |
|---|---|---|---|---|---|
| Lipid | carrying | Control | carrying | carrying | carrying |
| component | liposomes | liposomes | liposomes | liposomes | liposomes |
| DPPC | 25 | 40 | 35 | 35 | 35 |
| POPC | 25 | 30 | 30 | 30 | 30 |
| Cholesterol | 40 | 30 | 30 | 30 | 30 |
| DSPG | 10 | — | — | — | — |
| DSPE | — | — | 5 | — | — |
| DPDAP | — | — | — | 5 | — |
| DPTAP | — | — | — | — | 5 |
| Net charge | negative | neutral | neutral | neutral/ | positive |
| | | | | positive | |

For radioactive marking of the liposomes, 0.25 μCi/mg lipid (9.25 kBq/mg) [³H]cholesterylhexadecylether were added to the samples.

The solvent was removed from the resulting lipid mixture in a vacuum and then the sample was dried for 45 in the vacuum provided by an oil pump. The following buffer (pH 7.4) was used to prepare the liposomes (liposome buffer):

| | | |
|---|---|---|
| NaCl | 118 | mM |
| KCl | 4.74 | mM |
| KH₂PO₄ | 0.59 | mM |
| Na₂HPO₄ | 0.59 | mM |
| HEPES | 10 | mM |
| NaHCO₃ | 15 | mM |
| MgCl₂ | 1.18 | mM |

Prior to use, the liposome buffer was filtered through a membrane with a pore diameter of 220 μm. In order to resuspend the lipids, the desired quantity of liposome buffer was added to the dried lipid mixture and the sample was then stirred for 15 min at 40° C. at 50 rpm using a rotary evaporator. The resuspension of the lipids was accelerated substantially by the addition of 10-20 glass beads (710-1180 micron, Sigma). The quantity of liposome buffer added was selected to yield a lipid suspension with a concentration of 10-40 mg lipid/ml. The lipid suspension was centrifuged at 1000×g for 30 s and then allowed to stand at room temperature for 2 h. Finally, the multilamellar vesicles thus produced were extruded 12 times through a polycarbonate membrane (LFM-200, Milsch-Equipment, Laudenbach, Germany) with a pore size of 200 nm using a LiposoFast® extrusion apparatus (Milsch-Equipment), and then 21 times through two polycarbonate membranes (one on top of the other) (LFM-100, Milsch-Equipment) with a pore diameter of 100 nm. The liposomes were stored at 4° C. and used no later than 7 days after preparation.

2.2 Characterization of the Liposomes

2.2.1 Determination of Size

The size of the liposomes was determined with a ZetaSizer III (Malvern, England) applying the technique of photon correlation spectroscopy (Washington, C. (1992) “Particle size analysis in pharmaceutics and other industries”, (Rubinstein, M. H., ed.), Ellis Horwood, Ltd., London, England). Disposable plastic 3 ml-cuvettes containing approx. 0.3 mmol liposomal PC per ml cell buffer were used in the measurements. Each sample was measured in triplicate and the mean of the triplicate measurements was calculated.

2.2.2 Determination of the Phospholipid Concentration

The concentration of choline-containing phospholipids in the liposomal suspensions was determined by photometry (Stewart, J. C. M. in “Colorimetric determination of phospholipids with ammonium ferrothiocyanate”, Anal. Biochem. 104 (1979), p. 10-14). For this purpose, an ammonium ferrothiocyanate solution containing 27.03 g FeCl₃.6H₂O and 30.04 g NH₄SCN dissolved in 1 l distilled water, was prepared. The liposome suspension to be assayed for its phospholipid content was diluted to a total lipid content of approx. 2 mg/ml of liposome buffer. A total of 750 μl of the ammonium ferrothiocyanate solution described above were layered over 1 ml of CHCl₃ in a reaction vessel and then 25 μl of liposome dilution were added. Then, the two phases were mixed vigorously and subsequently separated by centrifugation at 2,500×g for 5 minutes. The upper aqueous phase resulting from centrifugation was removed. The chloroform phase was transferred to a 1 ml glass cuvette and the extinction of the solution at 485 nm was determined with a photometer. A sample containing no liposomes that was treated analogously was used as the blank. Five samples of each liposome suspension were measured and the mean of the values obtained was calculated. For calibration of the test, a phospholipid stock solution in CHCl₃ was prepared. The calibration was conducted with 6 different concentrations each of POPC and DPPC in the range of 0-150 nmol PC/ml CHCl₃. The test is linear in this range.

### 3. Reference Example 1

### Cascade Filtration of Liposomes

In cascade filtration, membranes differing in pore diameter are used to separate lipoproteins from blood. After plasma separation, the LDL particles are separated in accordance with the manufacturer's recommendations using a membrane with a pore diameter of 22 nm. In order to find out whether or not the approach of cascade filtration can be applied to liposomes, two consecutive filtration steps were used in an exemplary fashion to test whether liposomes can be filtered according to this procedure. In the first filtration step, a polycarbonate membrane with a pore diameter of 2 μm was used that is expected to be capable of separating blood cells from plasma. Polycarbonate was used in these tests as the material of choice because this filter material shows very low specific binding of the liposomes to the filter surface. In the second filtration step, a cellulose mixed ester filter with a pore diameter of 50 nm was used (both procured from Millipore GmbH, Eschborn, Germany) that is expected to retain the majority of the tested liposomes (liposomal diameter approx. 110 nm).

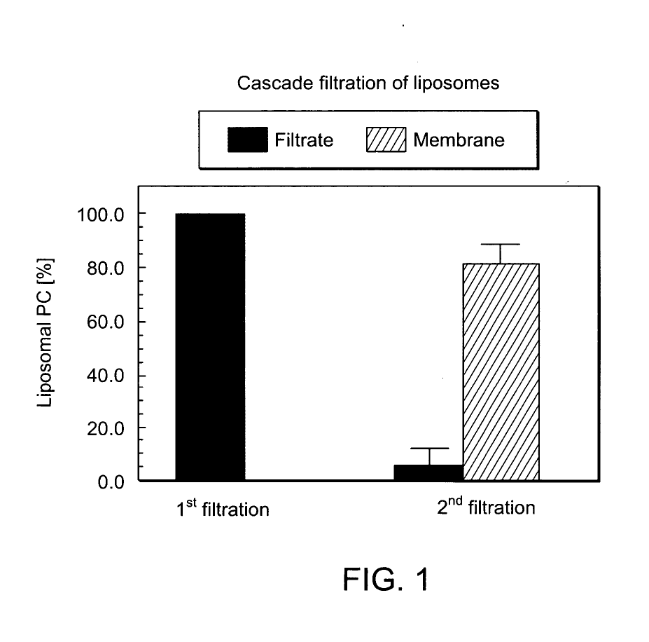

The separation of liposomes from the surrounding medium by means of cascade filtration was tested using a liposome suspension containing 0.5 mM liposomal PC. The test was performed on liposomes with a DSPG content of 10% and a mean size of approx. 110 nm. A modified version of the Millipore filtration technique of G. Brierley and R. L: O'Brian: “Compartimentation of heart mitochondria”, in J. Biol. Chem. 240 (1965), p. 4532-4539) was used for filtration of the liposomes. The filters had a diameter of 25 mm. The filtration was performed in a multiple filtration unit (Millipore GmbH, Eschborn, Germany). The vacuum applied to facilitate filtration was 900 mbar and 100 mbar in the first and second filtration step, respectively. The vacuum applied could be regulated by a three-way cock situated between the membrane pump and the filtration unit. The buffer used for filtration corresponded to the liposome buffer described above. After wetting with buffer, the polycarbonate membrane was placed in the filtration unit and 2 ml of liposome suspension were layered over the membrane at ambient pressure. Subsequently, the sample was filtered at reduced pressure. A 1 ml aliquot of the recovered filtrate was then applied at ambient pressure to a cellulose mixed ester membrane after the membrane had been wetted with buffer. Then, the liposome suspension was filtered again in a vacuum. An aliquot of the filtrate each was removed for assaying the amount of liposomal PC contained in the filtrate. After the filtration, the membranes were rinsed once with 1 ml of buffer and then transferred to a liquid scintillation vessel and dissolved by adding 750 μl Biolute-S® (Zinsser Analytik GmbH, Frankfurt, Germany). The radioactivity present in the filtrates and membranes was subsequently assayed as described above. The results obtained are shown in FIG. 1 and Table 2. Three individual measurements were carried out with one liposome preparation. FIG. 1 shows the amount of liposomal PC detected after filtration in the filtrate or on the filter membrane.

TABLE 2

| | |
|---|---|
| Filtrate | Membrane |

| | | | | |
|---|---|---|---|---|
| First filtration | 99.9% | (±0.1%) | 0.2% | (±0.1%) |
| Second filtration | 6.5% | (±6.1%) | 81.5% | (±7.2%) |

While very few, if any, of the liposomes are retained by the polycarbonate filter membrane with a pore diameter of 2 μm, the majority of the liposomes can be retained with a cellulose mixed ester membrane with a pore diameter of 50 nm. Thus, by means of using suitably selected membranes and adapting the filtration conditions, such as flow rate and filtration pressures, the technique of cascade filtration is generally suited for use for the separation of liposomes from blood.

### 4. Reference Example 2

### Precipitation of DSPE-Containing Liposomes

In order to demonstrate that, in principle, liposomes carrying charged groups on their surface are capable of interacting with oppositely charged polyions and in order to demonstrate the ensuing precipitability of such liposomes, in principle, as a result of this interaction heparin was used as polyanion and liposomes containing 5 mol % DSPE as liposomes. A liposome suspension containing 0.3 mM liposomal PC in liposome buffer was mixed at room temperature with an equal volume of 0.2 mM acetate buffer pH 4.85 containing 100 IE heparin/ml and 5 mM CaCl₂. The mixture was allowed to stand for 5 minutes followed by centrifugation for 10 min at 12,000×g. The supernatant was separated from the precipitate and the precipitate was dispersed in Ca²⁺-free liposome buffer containing 5 mM EGTA buffer.

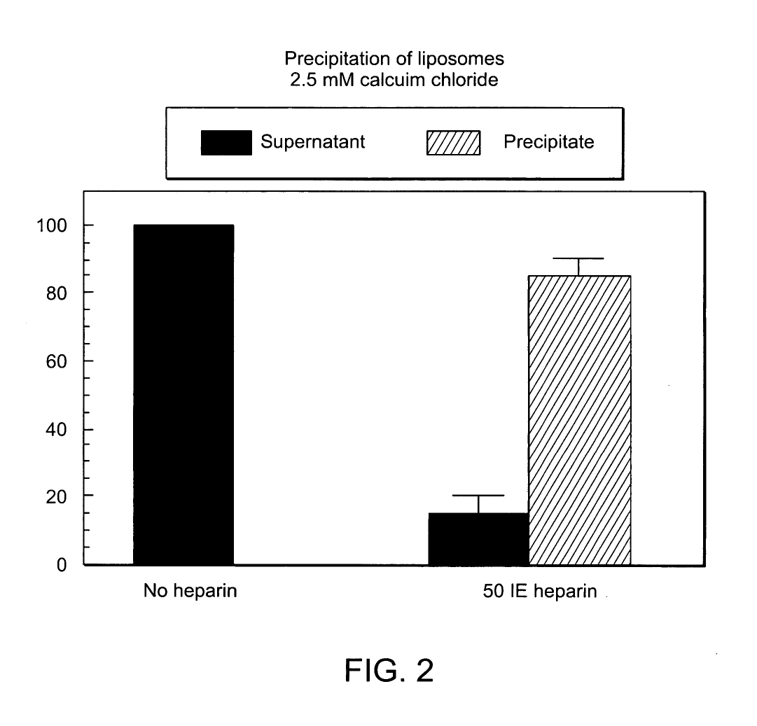

The radioactivity present in the supernatant versus precipitate was quantified as a measure of the quantity of liposomes in the supernatant and precipitate, respectively. In order to check the integrity of the precipitated liposomes, the size of the liposomes was investigated both prior and after precipitation. The results of precipitation are shown in FIG. 2 and quantified in Tables 3 and 4. One liposome suspension was used to perform 3 separate precipitations. FIG. 2 shows the corresponding means.

TABLE 3
Distribution of liposomal PC after
precipitation of DSPE-liposomes

| | Supernatant | Precipitate |
|---|---|---|
| Liposomal PC | 14.8% (±5%) | 85.2% (±5%) |

TABLE 4
Diameter of the liposomes before and after precipitation

| Before precipitation | After precipitation |
|---|---|
| 131.9 nm (±1.0 nm) | 136.6 nm (±0.1 nm) |

Using the data obtained, it was possible to show that liposomes carrying positively charged groups on their surface are capable of being precipitated by a polyanion in the presence of Ca²⁺. The precipitation occurred only in the presence of heparin as the polyanionic precipitating agent. The precipitation of liposomes was achieved under conditions as they are employed in HELP apheresis. Under these conditions, the precipitation is very efficient. In principle, the precipitation of liposomes is reversible, since the liposomes can be resuspended after centrifugation. Since the size of the liposomes remains virtually unchanged by precipitation, it can be presumed that the liposomes remain stable during precipitation and that the toxic substances possibly encapsulated within the liposomes are not released.

### 5. Reference Example 3

### Precipitation and Filtration of Liposomes Carrying Positive Net Charge

In the experiment illustrated in reference example 2, it was successfully shown that liposomes carrying positively charged groups on their surface can, in principle, be precipitated with polyanions. However, in the HELP procedure, the precipitated complexes of heparin and lipoproteins are not removed by centrifugation but by filtration of the corresponding serum through a polycarbonate membrane with a pore diameter of 450 nm. In order to determine whether or not complexes of heparin and lipoproteins can be filtered in analogy to the HELP procedure, liposomes were precipitated and then a filtration step was tested for its capacity to separate the liposomes from the surrounding medium. Liposomes containing 5% DPDAP or 5% DPTAP were used in these experiments.

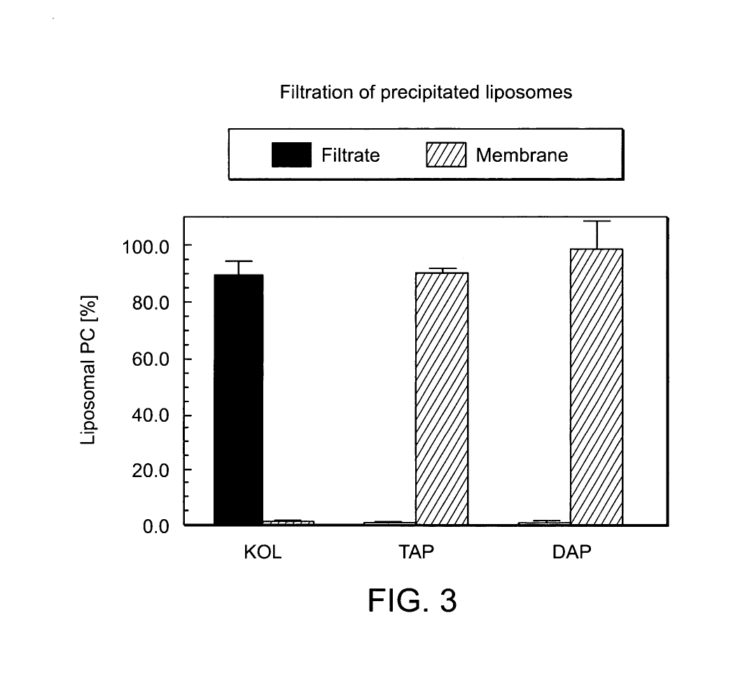

The liposomes were precipitated as described in reference example 2. However, the centrifugation step was replaced by filtration of 1 ml of the solution containing the precipitate according to the procedure described in reference example 1. Polycarbonate filters with a pore diameter of 400 nm (Millipore) were used for filtration. Filtration was performed at a vacuum of approx. 900 mbar. As before, an aliquot of the recovered filtrates was removed in order to assay the amount of liposomal PC present therein. The filter membranes were treated as described above in order to assay the amount of liposomal PC remaining on the membranes. The results obtained are shown in FIG. 3 and Table 5. The abbreviations are defined as follows:

KOL: liposomes carrying no charge,

TAP: liposomes containing 5 mol % permanently positively charged lipids,

DAP: liposomes containing 5 mol % of a lipid capable of being protonated.

One liposome preparation was used to perform two individual measurements.

TABLE 5
Filtration of precipitated liposomes

| | |
|---|---|
| Filtrate | Membrane |

| | | | | |
|---|---|---|---|---|
| KOL | 89.9% | (±4.3%) | 1.0% | (±0.5%) |
| TAP | 1.1% | (±0.1%) | 90.6% | (±1.3%) |
| DAP | 1.1% | (±0.6%) | 98.9% | (±9.9%) |

Liposomes carrying a positive net charge are capable of being precipitated by heparin and filtered under conditions as used in the HELP procedure. Accordingly, the procedure used in HELP apheresis, after appropriate adaptation, is, in principle, suitable for the elimination of liposomes from blood.

The HELP procedure is based on a reduction of the pH in the extracorporeal cycle. This allows the use of liposomes carrying lipids that are not charged at physiological pH, but become charged at lower pH such that they can be precipitated. The lipid, DPDAP, as used in this example, may serve as a lipid of this type. If liposomes with a permanent positive net charge at physiological pH are used, it may be possible to dispense with the pH reduction in the extracorporeal cycle.

### 6. Reference Example 4

### Adsorption Chromatography of Liposomes

The DALI procedure is based on a combination of gel permeation and adsorption chromatography. In order to demonstrate that liposomes can be removed, in principle, from a liposome suspension by adsorption chromatography, liposomes carrying either a negative net charge or a positive net charge were subjected to chromatography on a chromatography column containing Sephadex-bound heparin. DSPG-containing liposomes or DPDAP-containing liposomes were investigated in this experiment. An FPLC® system (Pharmacia, Freiburg, Germany) was used for chromatography. The chromatography column had a diameter of 16 mm and length of 35 mm and was operated at a flow rate of 0.5 ml/min. The sample applied to the column was 50 μl of a liposome suspension containing 0.5 mM liposomal PC. Fractions of 1 ml each were collected and the radioactivity present in these fractions was assayed as described above. A 0.2 mM acetate buffer pH 4.5 or a buffer containing 10 mM Hepes and 130 mM NaCl pH 9.0 was used for chromatography.

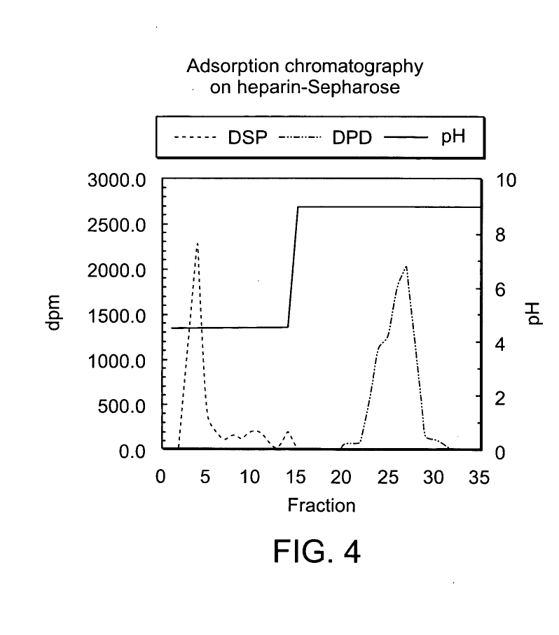

The chromatographic profile is shown in FIG. 4. Whereas the DSPG-containing liposomes with a negative net charge interacted very little, if any, with the heparin-containing column material and eluted within one column volume, the DPDAP-containing liposomes, which carry a positive net charge at pH 4.5, were quantitatively adsorbed to the column material. When the pH was increased to 9.0, the DPDAP-containing liposomes became protonated and were eluted from the column.

Therefore, liposomes can be separated from their surrounding medium by means of a suitable interaction, e.g. an electrostatic interaction, using the technique of adsorption chromatography. Because of the presence of an extracorporeal cycle, the conditions can be adapted to provide for optimal binding of the liposomes to the adsorption material.

1-31. (canceled)

32. A method for eliminating potentially toxic and/or harmful substances from a body fluid containing the same, wherein the potentially toxic and/or harmful substance is a therapeutic agent, the method comprising:
providing particles as drug carriers comprising a therapeutic agent, the particles being capable of binding, taking up and/or carrying the therapeutic agent;
providing a medical therapy with the particles;
and
removing the particles with the toxic and/or harmful substance from the body fluid in an extracorporeal step after medical therapy.

33. The method according to claim 32, wherein the removing step is performed in an extracorporeal step or in an exogeneous or extrinsic device by means of precipitation, cascade filtration, chromatography and/or adsorption.

34. The method according to claim 32, wherein the therapeutic agents are selected from the group consisting of cytostatic agents, antibiotics, antivirals, antimycotic agents, substances used in gene therapy, antibodies, interferons, cytokines, and radionuclides.

35. The method according to claim 32, further comprising recovering the particles after their removal from the body fluid.

36. The method according to claim 32, wherein the removing step comprises adjusting the content of the substance in the body fluid or adjusting the time course of a regimen of the therapeutic agent.

37. The method according to claim 36, further comprising determining the content of the substance in the body fluid after removing the particles or determining the content of substance in the particles after removing the particles.

38. The method according to claim 32, wherein the substance is an endogenous substance.
